(19) United States
(12) Patent Application Publication
(10) Pub. No.: US 2010/0057168 A1
(43) Pub. Date: Mar. 4, 2010

(54) HEATING SYSTEM

(75) Inventors: Alan P. Slade, Dundee (GB); Robert P. Keatch, Dundee (GB); Aneel Gill, Dundee (GB); Jill Belch, Dundee (GB); Faisel Khan, Dundee (GB)

Correspondence Address: GIFFORD, KRASS, SPRINKLE,ANDERSON & CITKOWSKI, P.C, PO BOX 7021, TROY, MI, 48007-7021, US

(73) Assignee: University of Dundee, Angus, Scotland (GB)

(21) Appl. No.: 12/515,599

(22) PCT Filed: Nov. 22, 2007
(86) PCT No.: PCT/GB2007/004461

§ 371 Date: Nov. 11, 2009

(30) Foreign Application Priority Data
Nov. 22, 2006 (GB) 0623225.0

(51) Int. Cl. A61F 7/00; H05B 3/00

(52) U.S. Cl. 607/99; 219/211

(57) ABSTRACT

A body part heating system (10) comprises a temperature sensor (28) adapted to measure the temperature of a body part, and a heater (34a, 34b) in communication with the temperature sensor (28). The heater (34a, 34b) is adapted to be activated to heat the body part in response to the temperature sensor (28) sensing the occurrence of a predefined thermal event such as a temperature drop below a predefined level. The heating system (10) may be in the form of a glove and may be used to treat the symptoms of Raynaud's phenomenon.

### FIELD OF THE INVENTION

The present invention relates to a heating system, and in particular to a body part heating system for treating or alleviating the symptoms of Raynaud's phenomenon.

### BACKGROUND TO THE INVENTION

Raynaud's phenomenon is a disorder that affects the blood vessels in the fingers, toes, ears and nose. This disorder is characterised by episodic vasospastic attacks usually in response to cold, that cause blood vessels in the digits and elsewhere to constrict. Raynaud's phenomenon can occur on its own, or it can be secondary to another condition such as systemic sclerosis or systemic lupus erythemotosus. Raynaud's phenomenon can cause a drastic reduction of the blood supply to a particular body part which may result in numbness, intense pain and tissue damage. In the most severe cases, ulceration and gangrene can occur.

Surveys have shown that Raynaud's phenomenon may affect 10% of the general population with women being nine times more likely to suffer from the disorder than men.

In addition to the above, people working in certain professions may be more vulnerable to a form of Raynaud's phenomenon. For example, it has been known for workers in plastics factories who are exposed to vinyl chloride to develop a scleroderma-like illness of which Raynaud's phenomenon can be a part. Workers who operate vibrating tools can develop another form of Raynaud's phenomenon called Hand Arm Vibration Syndrome (HVAS; formerly known as Vibration-Induced White Finger Disease).

Drugs are available to treat Raynaud's phenomenon. However, as a large proportion of patients with Raynaud's phenomenon are women of childbearing age, non-drug based therapies are preferred by both the clinician and patient.

One such non-drug based therapy includes the use of artificially heated gloves. However, although it has been shown that such devices offer some therapeutic benefit, their use is limited due to, for example, the bulky and heavy battery pack that must be carried if the gloves are to be used. In addition, many of the patients suffering with this condition are frail and elderly and are therefore less able to accommodate the heavy power source. Another problem is the fact that the gloves are heated uniformly across the whole of the glove causing those unaffected parts of the hand to overheat. Furthermore, existing gloves do not incorporate safety mechanisms which prevent the gloves from over-heating. Additionally, known gloves incorporate large power consuming resistive heaters wrapped in various patterns around the users hand. These heaters are not only power hungry but, as noted above, they have no temperature limiters and as such may reach dangerous temperatures.

All of the above noted and other factors result in gloves which are uncomfortable for the wearer and also extremely unfashionable. Thus, such therapeutic gloves are often not used by sufferers of Raynaud's phenomenon.

### SUMMARY OF THE INVENTION

According to a first aspect of the present invention, there is provided a body part heating system comprising:

a temperature sensor adapted to measure the temperature of a body part; and

a heater in communication with the temperature sensor and adapted to be activated to heat the body part in response to the temperature sensor sensing the occurrence of a predefined thermal event.

In one embodiment of the present invention, the thermal event may comprise a temperature drop below a predefined level. Alternatively, or additionally, the thermal event may comprise a predefined temperature gradient occurring between two or more regions of a body part.

Advantageously, in use, when the temperature sensor measures or senses that the predefined thermal event has occurred, such as a temperature drop of a body part below a predetermined level, then the heater will be activated to increase the temperature of the body part. Accordingly, the heating system of the present invention may advantageously be used to treat the symptoms of Raynaud's phenomenon.

Preferably, the heater is adapted to be deactivated when the temperature sensor senses that the predefined thermal event has ended or is eliminated, such as when a temperature increase above the predetermined level is detected.

Advantageously, the temperature sensor and heater may be separately formed. Alternatively, the temperature sensor and heater may be integrally formed.

Preferably, the heating system further comprises a control system. Preferably, the heater is in communication with the temperature sensor via the control system. The control system is preferably adapted to permit the heater to be activated and/or deactivated in response to the occurrence of the predefined thermal event being sensed by the temperature sensor. For example, the control system may be programmable to interpret data from the temperature sensor and to activate and deactivate the heater in accordance with said data.

The control system may comprise a microcontroller, such as a PIC microcontroller. Alternatively, or additionally, the control system may comprise a control circuit. At least one of the temperature sensor and heater may be incorporated within the control circuit. The control system may comprise a switching circuit adapted to permit the heater to be activated and/or deactivated in response to temperature changes sensed by the temperature sensor. The switching circuit may comprise a transistor adapted to be activated by an input signal initiated by the temperature sensor. The input signal from the temperature sensor may be communicated to the transistor via a suitable operational amplifier arrangement or other suitable signal conditioning device.

Preferably, the heating system comprises a wearable article adapted to be worn on a body part to be heated. The temperature sensor and heater are preferably mounted on the wearable article. The control system may be adapted to be mounted on the wearable article. Alternatively, the control system may be mounted separately of the wearable article. In one embodiment, one or both of the temperature sensor and heater are mounted on an internal portion of the wearable article. Alternatively, one or both of the temperature sensor and heater are mounted on an external portion of the wearable article. The wearable article may define a pocket or similar structure adapted to receive a component of the heating system, such as the temperature sensor, heater, control system or the like.

At least one of the temperature sensor and heater may be embedded in the wearable article. For example, at least one of the temperature sensor and heater may be stitched, threaded, woven, knitted, adhesively bonded or the like into the wearable article. Alternatively, or additionally, the temperature sensor and/or heater may be printed onto the wearable material, or alternatively onto a fabric or alternative substrate which is subsequently mounted on or secured to a wearable article. Alternatively, at least one of the temperature sensor, heater and any associated components of the heating system may be formed or mounted on a substrate and subsequently secured to a wearable article. The substrate may be flexible and may comprise a fabric. In a preferred embodiment, the substrate may comprise Tyvek™, a proprietary material from DuPont made from very thin, high density polyethylene fibres. Alternatively, the substrate may be substantially rigid, and may comprise a plastic material, composite material or the like.

Preferably, the wearable article is flexible. Preferably also, the wearable article is stretchable. The wearable article may comprise a fabric, which may be non-woven, woven, knitted, felted or the like. The wearable article may comprise nylon. The wearable article may comprise a waterproof material, and may comprise a breathable waterproof material, such as expanded PTFE.

Alternatively, or additionally, the wearable article may comprise a mouldable or formable material, such as a plastic or the like. The wearable article may comprise a synthetic material. Alternatively, or additionally, the wearable article may comprise a natural or non-synthetic material.

Preferably, the heater is flexible. Advantageously, the heater may comprise a carbon fibre heater. Alternatively, or additionally, the heater may comprise a flexible positive temperature coefficient (PTC) heater. Advantageously, the PTC heater may be printed onto fabric, wherein the fabric is adapted to be positioned on a body part to be heated. The fabric comprising the PTC may be subsequently secured to a wearable item.

Advantageously, a plurality of temperature sensors may be provided. Advantageously also, a plurality of heaters may be provided.

Preferably, the heating system comprises a power source. The power source may comprise an electrical cell. The electrical cell may be mounted adjacent to the heater and/or temperature sensor. Alternatively, the power source may be adapted to be mounted remotely from the heater and/or temperature sensor. The electrical cell may be rechargeable. For example, the electrical cell may be adapted to be coupled to a mains power source. Alternatively, the electrical cell may be rechargeable via a solar cell, or alternatively, or additionally, the electrical cell may be adapted to be recharged via a kinetic power supply. The power source may alternatively, or additionally, comprise a mains power source.

The electrical cell is preferably comprised in a battery pack. Preferably, the heating system comprises a wearable article and the battery pack may be adapted to be mounted on or within said wearable article. For example, the wearable article may comprise a glove and the battery pack may be mounted on or within a cuff region of the glove, in the palm region or in any other appropriate region which preferably achieves a comfortable fit for the user.

The battery pack may comprise a rigid casing. Alternatively, or additionally, at least a portion of a casing of the battery pack may be flexible. In this arrangement the flexible portion of the battery pack may be manipulated to conform to the shape of at least a portion of a body part. In this way the heating system may be readily adapted to suit various uses and users.

The battery pack may comprise a lithium ion based electrical cell. The battery pack may comprise a polymer electrolyte, which arrangement advantageously permits flexibility of the battery pack to be achieved.

Preferably, the heating system defines a plurality of zones, wherein each zone corresponds to a region of a body part. In one arrangement, each zone comprises at least one temperature sensor. Each zone may comprise at least one heater in communication with a respective temperature sensor. Each heater may therefore be adapted to be activated when an associated temperature sensor measures or senses the occurrence of the predefined thermal event, such as a temperature drop below a predetermined level. Advantageously, in use, this arrangement permits the heating system to provide selective heating, wherein heating is permitted only in those zones or regions of a body part in which the thermal event occurs. This therefore beneficially prevents overheating in zones where the thermal event does not exist. Furthermore, activating heaters in only those zones where the thermal event occurs assists to conserve power. The thermal event may be common to all zones. Alternatively, the thermal event may differ between one or more zones.

The heater in each zone may be adapted to be activated independently from each other. Alternatively, or additionally, the heater in each zone may be adapted to be activated simultaneously.

In one embodiment of the present invention, a heater in one zone may be adapted to be activated when a temperature sensor in another zone senses or measures the occurrence of the thermal event, such as temperature drop, in said other zone. For example, where the temperature in one zone falls below the predetermined level, the heater in an adjacent zone may additionally be activated for a predetermined time or until a predetermined temperature increase in achieved. Heating the adjacent zones may contribute to raising the temperature in said zones above the predetermined level.

The heating system may comprise at least one heater adapted to extend across two or more zones, wherein the heater is in communication with a temperature sensor in each of said zones. The temperature sensors may be adapted to detect a predefined thermal gradient occurring between the zones. When such a predefined thermal gradient is detected then the heater extending across the respective zones may be activated to seek to minimise the thermal gradient.

In one embodiment, each zone may comprise a separate control system. Alternatively, a common control system may be provided for two or more zones.

Preferably, the heating system comprises a temperature limiter adapted to limit the temperature of the heater. This arrangement therefore permits the maximum temperature achievable to be capped to prevent or minimise the risk of burning a user. The temperature limiter may be integrally formed with the heater or may be an inherent feature or property of the heater. Alternatively, the temperature limiter may be provided within a control system. Preferably, the temperature limiter is adapted to limit the temperature of the heaters to less than 50° C., and more preferably less than 40° C.

The heating system may be adapted to be activated for a predetermined time. The heating system may comprise a timing circuit or the like.

The heating system may be adapted to be manually operated which may permit a user to have a degree of control over the heating provided by the system.

In a preferred embodiment, the heating system is adapted for use in heating a hand, and in particular one or more digits of the hand. The heating system may therefore be adapted to be worn on the hand of a user. The heating system may comprise a glove, mitten or the like. Alternatively, the heating system may be adapted for use in heating other body parts, such as the ears, head, nose, feet, toes, or the like. The heating system may comprise or be mounted on or in a hat, scarf, blanket or the like.

The heating system may be adapted to be incorporated into outdoor clothing, such as mountaineering clothing, skiing clothing or the like.

The heating system may be washable.

According to a second aspect of the present invention, there is provided a method of regulating the temperature of a body part, said method comprising the steps of;

mounting a temperature sensor adjacent a body part;

mounting a heater adjacent the body part, wherein the heater is in communication with the temperature sensor; and

sensing the temperature of the body part and activating the heater when the occurrence of a predefined thermal event is sensed.

The predefined thermal event may comprise a drop in temperature below a predetermined level. Alternatively, or additionally, the predefined thermal event may comprise a thermal gradient within the body part.

Preferably, the method comprises the steps of:

mounting a first temperature sensor adjacent a first region of the body part and a second temperature sensor adjacent a second region of a body part;

sensing the temperature of each region of the body part; and

activating the heater in accordance with the sensed temperatures in the first and second regions of the body part.

In this arrangement the heater may be activated in response to a predefined thermal gradient being created between the first and second regions of the body part.

The method may comprise the steps of mounting a heater across the first and second regions of the body part. Alternatively, or additionally, the method may comprise the steps of providing a first heater adjacent the first region of the body part and a second heater adjacent the second region of the body part, wherein the heaters are in communication with the first and second temperature sensors respectively.

According to a third aspect of the present invention, there is provided a heated glove comprising:

a digit portion;

a temperature sensor mounted within the digit portion; and

a heater mounted within the digit portion and in communication with the temperature sensor;

wherein, in use, the heater is adapted to be activated to heat a digit when the temperature sensor senses the occurrence of a predefined thermal event.

Preferably, the digit portion defines a plurality of zones, wherein each zone corresponds to a region of a digit of a user. Preferably, each zone comprises at least one temperature sensor. Each zone may comprise at least one heater in communication with a respective temperature sensor. Each heater may be adapted to be activated when an associated temperature sensor measures or senses the occurrence of the predefined thermal event, such as a temperature drop below a predetermined level.

Alternatively, at least one heater may extend across two or more zones, wherein the heater may be activated in response to a predefined thermal gradient being sensed or measured by the respective temperature sensors in the zones.

Preferably, the glove is adapted for treating or alleviating the symptoms of Raynaud's phenomenon.

According to a fourth aspect of the present invention, there is provided a heating system adapted to be mounted on a wearable item, said heating system comprising:

a support:

a temperature sensor mounted on the support and adapted to measure the temperature of a body part; and

a heater mounted on the support and in communication with the temperature sensor, wherein the heater is adapted to be activated to heat the body part in response to the temperature sensor sensing the occurrence of a predefined thermal event.

Accordingly, the heating system may be mounted on or within an existing wearable item, such as a glove, scarf, hat or the like. The support may be adapted to be secured to the wearable item, such as by stitching, bonding hook and loop fasteners, buttoning or the like. Alternatively, the support may be adapted to be positioned within a pocket or the like formed in the wearable item.

### BRIEF DESCRIPTION OF THE DRAWINGS

These and other aspect of the present invention will now be described, by way of example only, with reference to the accompanying drawings, in which:

FIG. 1 is a diagrammatic representation of a body part heating system, in particular a heated glove, in accordance with an embodiment of the present invention;

FIG. 2 is a diagrammatic view of a finger of the glove shown in FIG. 1;

FIG. 3 is a diagrammatic view of a control system for use with the glove of FIG. 1;

FIG. 4 is a schematic of a control circuit suitable for use with the glove of FIG. 1;

FIG. 5 is a diagrammatic representation of the heated glove of FIG. 1, shown with a battery pack in accordance with an embodiment of the invention;

FIG. 6 is a diagrammatic representation of the heated glove of FIG. 1, shown with a battery pack in accordance with an alternative embodiment of the invention; and

FIG. 7 is a diagrammatic view of a finger of a heated glove in accordance with an alternative embodiment of the present invention.

### DETAILED DESCRIPTION OF THE DRAWINGS

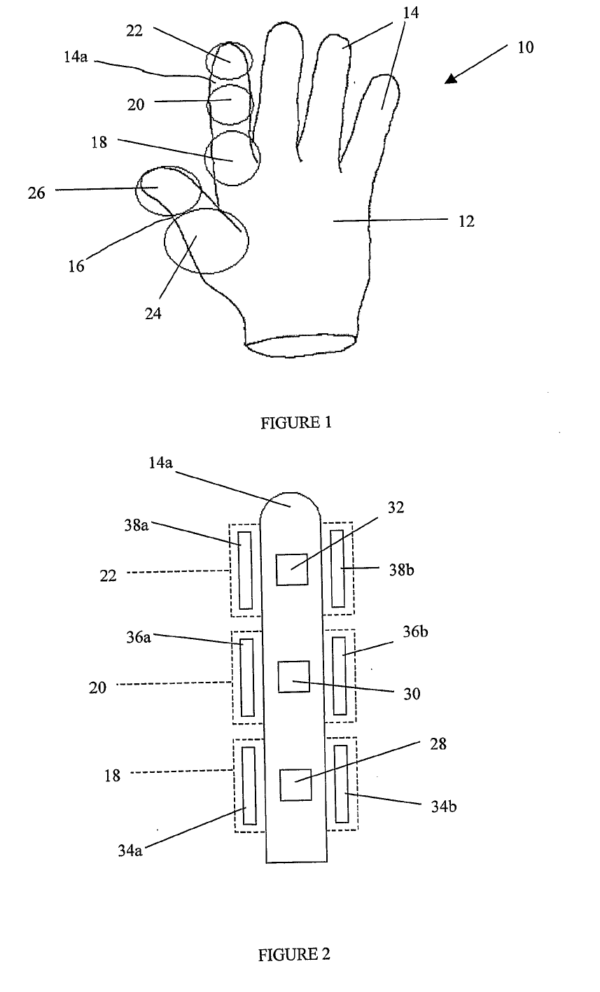

Reference is first made to FIG. 1 of the drawings in which there is shown a body part heating system, in this case a heated glove, in accordance with an embodiment of the present invention. The glove, generally identified by reference numeral 10, is of a conventional shape and as such comprises a hand portion 12 from which extends four finger portions 14 and a thumb potion 16. As will be discussed in further detail below, the glove 10 is adapted for use in selectively heating portions of a user's hand, and is particularly adapted for use in alleviating the symptoms of Raynaud's phenomenon.

In the embodiment shown, each finger portion 14 defines three separate regions, as exemplified on index finger portion 14a, specifically a proximal region 18, middle region 20 and distal region 22. The thumb portion 16 defines two separate regions, namely a proximal region 24 and distal region 26. Each region 18, 20, 22, 24, 26 comprises a temperature sensor and a pair of heaters (not shown in FIG. 1) in electrical communication with the temperature sensor, wherein the heaters are adapted to be activated when the associated temperature sensor senses or measures the occurrence of a predefined thermal event, such as a temperature drop below a predetermined level or the creation of an adverse thermal gradient between regions of a finger portion 14. For example, when in use, if the temperature sensor in the distal region 22 of a finger 14a senses a temperature drop in that region of a user's finger, the associated heaters will be activated in order to raise the temperature of the region 22. The heaters may be adapted to be activated when the region of the body part falls below, for example, 20° C., more preferably below 25° C., and most preferably below 28° C.

Accordingly, the glove 10 is capable of selectively heating regions of a user's hand. Thus, when a region of a user's hand experiences a temperature drop as a result of an attack of Raynaud's phenomenon, the glove 10 will react by heating only the affected region in order to raise its temperature and minimise discomfort and long-term damage. This therefore prevents overheating of other portions of the user's hand which have not been affected by the Raynaud's attack, contrary to existing therapeutic heated gloves which continuously heat the whole of the user's hand.

An enlarged diagrammatic view of the index finger portion 14a of FIG. 1 is shown in FIG. 2, reference to which is now made. Each region 18, 20, 22 comprises a respective temperature sensor 28, 30, 32 and a respective pair of heaters 34a, 34b, 36a, 36b, 38a, 38b. In the preferred embodiment shown, the temperature sensors 28, 30, 32 are thermistors and the heaters 34a, 34b, 36a, 36b, 38a, 38b are flexible positive temperature coefficient (PTC) heaters. The temperatures sensors 28, 30, 32 and heaters 34a, 34b, 36a, 36b, 38a, 38b may be attached directly to the glove 10, for example by stitching. Alternatively, the components may be mounted or secured on a substrate and then secured to the glove 10.

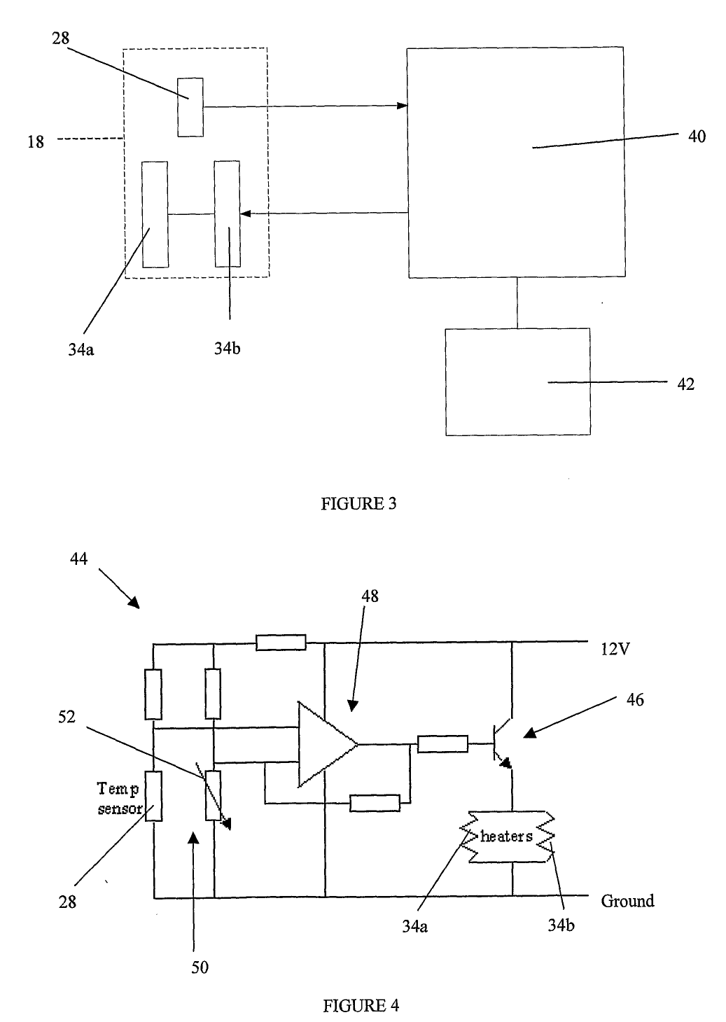

The glove 10 further comprises a control system which in one embodiment comprises a microcontroller, as diagrammatically shown in FIG. 3, reference to which is now made. The microcontroller, generally identified by reference numeral 40, is a PIC microcontroller and is shown associated with the proximal region 18 of the index finger 14a of the glove. However, the microcontroller 40 may also be associated with other regions of the same index finger 14a or alternatively with other regions of the fingers 14 and thumb 16 of the glove 10. Accordingly, the glove 10 may comprise one or more microcontrollers. In the embodiment shown in FIG. 3 the microcontroller 40 is programmable to interpret data from the temperature sensor 28 and to activate and deactivate the heaters 34a, 34b in accordance with said data, and preferably in accordance with predetermined conditions. Specifically, the microcontroller 40 is programmable to control or set a predetermined temperature level, below which the heaters 34a, 34b are activated, and above which the heaters 34a, 34b are deactivated. The microcontroller 40 may also be programmable to limit the maximum temperature of the heaters 34a, 34b, preferably to around 40° C., to prevent burning the user's hand.

A battery 42, preferably a lithium ion battery is electrically coupled to the microcontroller 40 to provide power to the glove 10. Suitable battery arrangements in accordance with embodiments of the invention are discussed in further detail below.

In an alternative embodiment, the control system of the glove 10 may comprise a control circuit, as shown in FIG. 4, reference to which is now made. The control circuit, generally identified by reference numeral 44, comprises the temperature sensor 28 and heaters 34a, 34b of region 18. Accordingly, in a preferred embodiment each region of the glove comprises a respective control circuit. In the embodiment shown the temperature sensor 28 is a thermistor and the heaters 34a, 34b are PTC heaters. The temperature sensor 28 and heaters 34a, 34b, along with associated wiring, may be directly mounted on the glove 10, and may be embedded within the fabric of the glove 10, for example by stitching, weaving or the like.

The control circuit 44 is a switching circuit and comprises a transistor 46 adapted to be activated to switch the heaters 34a, 34b on and off in response to an input signal initiated by the temperature sensor 28. The input signal from the temperature sensor 28 is communicated to the transistor via a suitable operational amplifier arrangement 48. The characteristics of the temperature sensor 28 and the signal generated and communicated to the transistor via the operational amplifier 48 is selected by virtue of a resistor arrangement, generally identified by reference numeral 50. The resistor arrangement 50 is adapted to generate an output sufficient to activate the transistor 46 when the temperature sensor 28 is exposed to a predetermined temperature level or range. Preferably, the resistor arrangement 50 comprises a variable resistor 52 adapted to permit the control system/circuit to be reactive when the temperature sensor 28 is exposed to a predetermined temperature level or range.

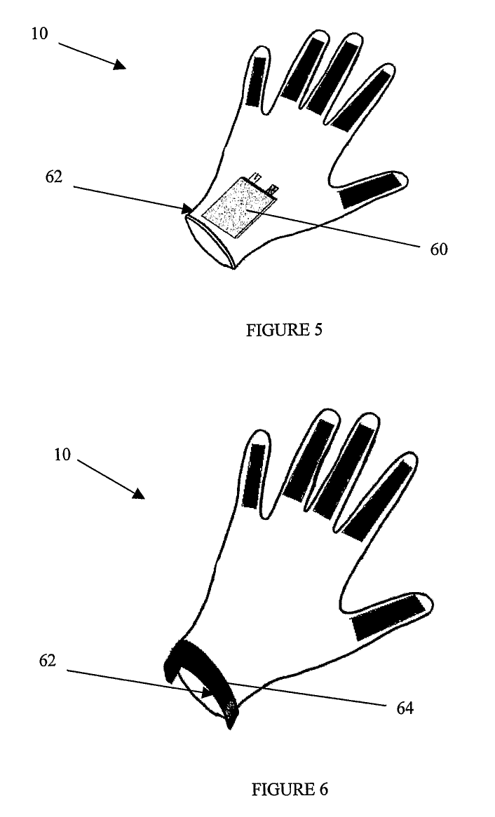

The arrangement shown in FIG. 4 is powered by a 12V battery (not shown), which may be a lithium ion battery. Arrangements of battery forms suitable for use with embodiments of the present invention are shown in FIGS. 5 and 6, reference to which is now made.

Referring first to FIG. 5, a lithium ion battery pack 60 comprising a rigid casing is shown positioned within a cuff region 62 of the glove. In this arrangement, the battery is of a compact form permitting integration of the battery pack 60 into the glove 10 while minimising discomfort to a user.

In the alternative arrangement shown in FIG. 6, a polymer lithium ion battery pack 64 is positioned in the cuff region 62 of the glove 10. The battery pack 64 comprises a flexible casing such that the battery pack 64 may adopt the form of the cuff region 64 of the glove 10.

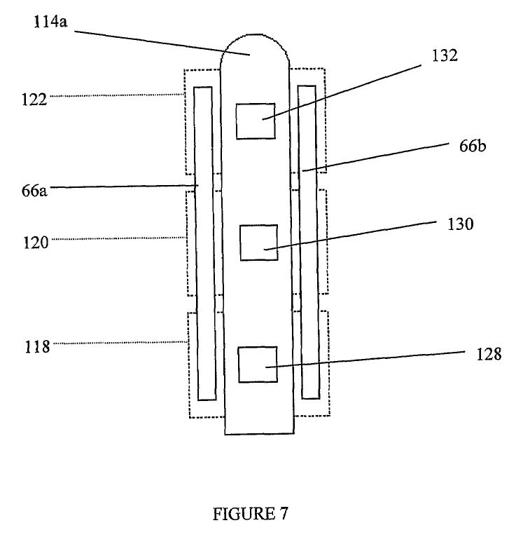

A diagrammatic view of a finger portion of a heated glove in accordance with an alternative embodiment of the present invention is shown in FIG. 7, reference to which is now made. The embodiment shown in FIG. 7 is similar to that shown in FIG. 3 and as such like components and features share like reference numerals, incremented by 100. Accordingly, the finger portion 114a defines three regions, a proximal region 118, a middle region 120 and a distal region 122. Each region comprises a temperature sensor 128, 130, 132, preferably thermistors. A pair of heaters 66a, 66b, preferably PTC heaters, extend across all three regions 118, 120, 122. In use, the temperature sensors 128, 130, 132 measure the temperature of a corresponding region of a body part such that the temperature difference or gradient between the regions may be obtained. When the temperature gradient exceeds a predefined level then the heaters 66a, 66b are activated until the temperature gradient is modified to within acceptable limits. For example, the heaters 66a, 66b may be activated when a temperature difference or gradient of 5° C. or more is detected. Accordingly, in this arrangement the heaters may selectively be used to alleviate an attack of Raynaud's phenomenon.

In an alternative arrangement, a heater may be provided in each zone 118, 120, 122, wherein the heaters are adapted to be activated simultaneously to thus provide concurrent heating in each zone. This arrangement therefore permits flexibility of the finger portion 114a from being impeded by a single heater extending across two or more zones.

The present invention has significant advantages over existing heated glove systems used to alleviate the symptoms of Raynaud's phenomenon. For example, the glove of the present invention only heats those regions of the user's hand which suffer an attack of Raynaud's phenomenon. As such, the present invention prevents overheating of the user's hand. Additionally, by providing selective heating the power consumption of the glove may be minimised, thus permitting the use of a smaller and more compact power supply. This, in combination with the use of e-textile components, minimises the size of the glove and ancillary equipment which results in a smaller glove which is more likely to be adopted and frequently used by a sufferer of Raynaud's phenomenon. Additionally, the glove of the present invention incorporates temperature limiters such that the heaters are not permitted to heat the user's hand above a certain level. Of course, other advantages of the present invention will be appreciated by those of skill in the art.

It should be understood that the embodiments described above are merely exemplary and that various modification may be made thereto without departing from the scope of the invention. For example, the principle of the invention may be utilised in other wearable items, such as hats, scarves, ear warmers, socks, blankets or the like. Additionally, the glove, or other wearable item, may be adapted to selectively heat any suitable number of regions of a body part. Furthermore, the glove is not limited for use in treating or addressing Raynaud's phenomenon. For example, the heating system of the present invention may be utilised in outdoor clothing. Additionally, the heating system may be used in first aid equipment, for example for treating conditions such as hypothermia, frostbite or the like where controlled heating is beneficial.

1. A body part heating system comprising:
a temperature sensor adapted to measure the temperature of a body part; and
a heater in communication with the temperature sensor and adapted to be activated to heat the body part in response to the temperature sensor sensing the occurrence of a predefined thermal event.

2. The system of claim 1, wherein the thermal event comprises a temperature drop below a predefined level.

3. The system of claim 1, wherein the thermal event comprises a predefined temperature gradient occurring between two or more regions of a body part.

4. The system of claim 1, further comprising a control system.

5. The system of claim 4, wherein the heater is in communication with the temperature sensor via the control system.

6. The system of claim 4, wherein the control system comprises a microcontroller.

7. The system of claim 4, wherein the control system comprises a control circuit.

8. The system of claim 1, further comprising a wearable article adapted to be worn on a body part to be heated.

9. The system of claim 8, wherein the temperature sensor and heater are mounted on the wearable article.

10. The system of claim 8, wherein the temperature sensor and/or heater are embedded in the wearable article.

11. The system of claim 8, wherein the temperature sensor is printed onto the wearable material.

12. The system of claim 1, wherein the temperature sensor and/or the heater are formed on a substrate and subsequently secured to a wearable article.

13. The system of claim 1, wherein the heater comprises a positive temperature coefficient (PTC) heater.

14. The system of claim 13, wherein the PTC heater is printed onto fabric, wherein the fabric is adapted to be positioned on a body part to be heated.

15. The system of claim 1, further comprising a power source.

16. The system of claim 15, wherein the power source comprises an electrical cell.

17. The system of claim 16, wherein the electrical cell is comprised in a battery pack.

18. The system of claim 17, wherein the battery pack comprises a rigid casing.

19. The system of claim 16, wherein at least a portion of a casing of the battery pack is flexible.

20. The system of claim 1, wherein the heating system defines a plurality of zones, wherein each zone corresponds to a region of a body part.

21. The system of claim 20, wherein each zone comprises at least one temperature sensor.

22. The system of claim 21, wherein the temperature sensors are adapted to detect a predefined thermal gradient occurring between the zones.

23. The system of claim 20, wherein each zone comprises at least one heater.

24. The system of claim 20, comprising at least one heater adapted to extend across two or more zones.

25. The system of claim 1, further comprising a temperature limiter adapted to limit the temperature of the heater.

26. The system of claim 25, wherein the temperature limiter is integrally formed with the heater.

27. The system of claim 1, comprising a glove.

28. A method of regulating the temperature of a body part, said method comprising the steps of:
mounting a temperature sensor adjacent a body part;
mounting a heater adjacent the body part, wherein the heater is in communication with the temperature sensor; and
sensing the temperature of the body part and activating the heater when the occurrence of a predefined thermal event is sensed.

29. The method of claim 28, comprising the steps of:
mounting a first temperature sensor adjacent a first region of the body part and a second temperature sensor adjacent a second region of a body part;
sensing the temperature of each region of the body part; and
activating the heater in accordance with the sensed temperatures in the first and second regions of the body part.

30. A heated glove comprising:
a digit portion;
a temperature sensor mounted within the digit portion; and
a heater mounted within the digit portion and in communication with the temperature sensor;
wherein, in use, the heater is adapted to be activated to heat a digit when the temperature sensor senses the occurrence of a predefined thermal event.

31. A heating system adapted to be mounted on a wearable item, said heating system comprising:
a support;
a temperature sensor mounted on the support and adapted to measure the temperature of a body part; and
a heater mounted on the support and in communication with the temperature sensor, wherein the heater is adapted to be activated to heat the body part in response to the temperature sensor sensing the occurrence of a predefined thermal event.
